(19) United States
(12) Patent Application Publication
(10) Pub. No.: US 2005/0282834 A1
(43) Pub. Date: Dec. 22, 2005

(54) Compositions, devices and methods for treating cardiovascular disease

(76) Inventors: Fady Malik, Burlingame, CA (US); Gustave Bergnes, Pacifica, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/147,406

(22) Filed: Jun. 7, 2005

Related U.S. Application Data
(60) Provisional application No. 60/578,755, filed on Jun. 9, 2004.

(51) Int. Cl. A61K 31/517

(52) U.S. Cl. 514/266.3

(57) ABSTRACT

In situ drug-delivering medical devices, materials and associated compounds, pharmaceutical compositions and methods are disclosed for the treatment of diseases of proliferating cells, particularly atherosclerosis and restenosis.

This application claims the benefit of U.S. Provisional Patent Application No. 60/578,755, filed Jun. 9, 2004; which is incorporated herein by reference for all purposes.

The invention relates to in situ drug-delivering medical devices, materials (such as drug-coated and drug-eluting stents) and associated compounds, pharmaceutical compositions and methods for the treatment of diseases of proliferating cells, particularly atherosclerosis and restenosis.

Abnormal proliferation of cells can give rise to disease states, such as cancer, cardiovascular disease, autoimmune disease, arthritis, graft rejection, inflammatory bowel disease, and proliferation induced after medical procedures (e.g., surgery, angioplasty, and the like), which can in turn be treated by modulating such proliferation. The role of cellular hyperproliferation in cancer is well known. In autoimmune diseases, such as arthritis, as well as in graft rejection, a proliferation of T-cells and/or antibody-producing B cells can result in the erroneous destruction of beneficial tissues. Proliferating cells need not, however, be in a hyper or hypo proliferation state (abnormal state) in order to warrant modulation. For example, during wound healing, the cells may be proliferating “normally”, but proliferation enhancement may be desired.

Vascular disease may result from undesirably proliferating cells in the blood vessels that supply vital organs, particularly the heart, as a result of the disease processes of arteriosclerosis, atherosclerosis, and restenosis. Arteriosclerosis results in degenerative changes and fibrosis in small arteries (arterioles), while atherosclerosis is a disease of medium- and large-sized muscular and elastic arteries such as the coronary arteries, the aorta, the carotid, major arteries supplying the brain, and arteries supplying the peripheral vasculature, particularly, the leg arteries, such as the iliac and femoral arteries. Restenosis is the local proliferation of cells that occurs after a vascular intervention is performed to correct a vascular stenosis resulting from atherosclerosis. This proliferation of cells can lead to the recurrence of the vascular stenosis.

The main pathogenic process in these vascular diseases is the significant narrowing of blood vessels through a build-up of lesions (or “plaque”) in one or more arteries. In the peripheral vasculature, this can lead to gangrene and loss of function of the extremities. When coronary arteries narrow more than 50-70%, the blood supply beyond the plaque becomes inadequate, e.g., to meet the increased oxygen demand during exercise. Lack of oxygen (or ischemia) in the heart muscle usually causes chest pain (or “angina”) in most patients. However, 25% of patients experience no chest pain at all despite documented ischemia; these patients have “silent angina” and have the same risk of heart attack as those with angina. When arteries are narrowed in excess of 90-99%, patients often have angina even when at rest. In those cases where a blood clot forms on the plaque, the artery can become completely blocked, causing death of the associated heart muscles.

Pharmaceutical and surgical treatments for vascular diseases have achieved varying degrees of success. Attempts to treat atheroma include efforts designed to lower plasma cholesterol levels through medication. When atheromas are symptomatic, vascular interventions such as angioplasty, atherectomy, endarterectomy, coronary or peripheral artery bypass grafting are considered. Balloon angioplasty [also termed percutaneous transluminal coronary angioplasty (“PTCA”)], has been used to enlarge narrowed arteries. In this procedure, a catheter with a deflated balloon on its tip is passed into the narrowed part of the artery. The balloon is then inflated, and the narrowed area widened. Limitations of balloon angioplasty include abrupt vessel closure or recoil after balloon expansion resulting in an adverse outcome or suboptimal final vessel luminal diameter. In a significant percentage of patients, the stenosis returns as the vessel heals over the course of 3-6 months, a process known as restenosis.

Stents are expandable supports placed inside arteries and have solved many of the shortcomings of balloon angioplasty such as vessel recoil and abrupt closure. The stent is collapsed to a small diameter, placed over an angioplasty balloon catheter, and maneuvered into the constricted area. When the balloon is inflated, the stent expands in place and forms a rigid support to hold the artery open. This scaffolding ensures allows for optimal expansion of the vessel stenosis. Stents reduce the incidence of restenosis by about 50% as compared to balloon angioplasty; however, depending on vessel size, lesion length, and whether the patient has diabetes, restenosis can occur in up to 30% of patients. This process is termed in stent restenosis and is due to excessive cellular proliferation at the stent implantation site. The pathologic process is neointimal proliferation. Despite this problem of in stent restenosis, stent implantation represents an improvement over balloon angioplasty and is utilized in the majority of coronary vascular interventions.

Despite its advantages, about 20% of patients receiving stents are required to undergo a repeat vessel intervention to increase coronary artery blood flow. Sometimes intravascular radiation is used to prevent the process of restenosis from reoccurring. Occasionally, the process of restenosis is recalcitrant to radiation or repeat interventions and patients will require a surgical procedure, coronary artery bypass grafting.

A variety of agents have been tried, both as orally taken drugs and as agents coated on and released from the stents themselves. The majority have largely failed to significantly alter post-angioplasty restenosis in human trials including, e.g., antiplatelet agents, anticoagulants, thromboxane antagonists, prostanoids, calcium channel blockers, ace inhibitors, antiproliferative growth factor inhibitors, lipid lowering agents, corticosteroids, and non-steroidal antiinflammatory agents. Two agents have proven successful in reducing the rate of in-stent restenosis when coated on the stent itself. Both of these agents, rapamycin and taxol, have anti-proliferative effects. Despite their efficacy, the restenosis still occurs in a significant percentage of lesions, underlining the continued need to explore new mechanisms to prevent restenosis. Over one million coronary interventions are performed in the United States every year so that even a small incidence of restenosis would result in a significant number of repeat procedures.

One novel anti-proliferative mechanism entails selective inhibition of mitotic kinesins, enzymes that are essential for assembly and function of the mitotic spindle. The mitotic spindle is responsible for distribution of replicate copies of the genome to each of the two daughter cells that result from cell division. Disruption of the mitotic spindle can result in inhibition of cell division, and the induction of cell death. Mitotic kinesins play essential roles during all phases of mitosis. These enzymes are “molecular motors” that transform energy released by hydrolysis of ATP into mechanical force that drives the directional movement of cellular cargoes along microtubules. The catalytic domain responsible for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins that have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human organisms, bundle antiparallel microtubules and slide them relative to one another, thus forcing the two spindle poles apart. KSP may also mediate in anaphase B spindle elongation and focussing of microtubules at the spindle pole.

Human KSP (also termed HsEg5) has been described [Blangy, et al., Cell, 83:1159-69 (1995); Whitehead, et al., Arthritis Rheum., 39:1635-42 (1996); Galgio et al., J. Cell Biol., 135:339-414 (1996); Blangy, et al., J. Biol. Chem., 272:19418-24 (1997); Blangy, et al., Cell Motil Cytoskeleton, 40:174-82 (1998); Whitehead and Rattner, J. Cell Sci., 111:2551-61 (1998); Kaiser, et al., JBC 274:18925-31 (1999); GenBank accession numbers: X85137, NM004523 and U37426], and a fragment of the KSP gene (TRIP5) has been described [Lee, et al., Mol Endocrinol., 9:243-54 (1995); GenBank accession number L40372]. Xenopus KSP homologs (Eg5), as well as Drosophila KLP61 F/KRP1 30 have been reported.

The sustained, controlled and/or localized delivery of therapeutic agents has been accomplished through a variety of formulations, materials and devices. These have ranged from transdermal patches and subcutaneous implants to surgical materials and devices, including: simple cylinders, spheres (microspheres, nanospheres, pellets), pliable moldable solids, fibers, and drug-bearing reservoirs and coatings associated with materials and devices otherwise intended for placement (e.g., stents, angioplasty balloons, contact lenses, brachytherapy seeds, orthopedic and dental bone dowels, prostheses such as breast implants, surgical sponges, wound dressings and gel-forming fluids for placement in body cavities). See, for example, U.S. Pat. Nos. 5,084,050; 5,551,954; 5,676,963; 5,788,979; 5,972,366; 6,153,252; 6,261,583; 6,346,272 as well as the background information there-discussed. U.S. Pat. No. 6,273,913 describes an intra vascular stent for the delivery of a therapeutic agent (particularly, rapamycin) from one or more reservoirs in the stent body, for the prevention of restenosis. A biocompatible coating or membrane is described to control drug diffusion from the reservoirs. The patent also describes drug delivery from micropores in the stent body or drug that is mixed/bound to a polymer coating applied on the stent.

There are two primary categories of drug-delivery stents: “drug-coated” stents (which allow for the placement and local delivery of a drug at an implantation site) and “drug-eluting” stents (which allow for the active, controlled release of a drug from an implantation site). Such in-situ drug delivery can greatly facilitate the bioavailability and targeting of an active agent. One example of a drug-coated stent employs heparin, an anti-coagulant drug. Another drug-eluting stent employs sirolimus (rapamycin), an immunosuppresive drug, and has been reported to significantly reduce the incidence of restenosis; recent reports of blood clots at the site of stent implantation in patients receiving this device suggest a need for further improvements in drug-eluting stent materials and/or active agents.

The present invention provides in situ drug-delivering medical devices and materials (such as drug-coated and drug-eluting stents), compounds, pharmaceutical compositions and methods for the treatment of diseases of proliferating cells, particularly atherosclerosis and restenosis. The compounds are KSP inhibitors, particularly inhibitors of human KSP.

In one aspect, the invention relates to a medical device/material having an effective amount of at least one KSP inhibitor, such as at least one chemical entity chosen from compounds represented by Formula I: 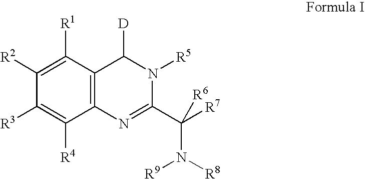
and pharmaceutically acceptable salts thereof, where:
R¹, R², R³ and R⁴ are independently hydrogen, hydroxy, halo, optionally substituted alkyl, optionally substituted alkoxy, optionally substituted amino, optionally substituted aryl, acyl, nitro, or cyano;
R⁵ is optionally substituted alkyl or optionally substituted aryl;
R⁶ and R⁷ are independently hydrogen, optionally substituted alkyl or optionally substituted aryl;
R⁸ is optionally substituted alkyl or optionally substituted aryl;
R⁹ is hydrogen, —C(O)—R¹⁰, —CH₂—R¹⁰, —C(O)—NH—R¹⁰, —S(O)₂—NH—R¹⁰, —C(O)₂—R¹¹, or —S(O)₂—R¹¹, in which:
   R¹⁰ is hydrogen, optionally substituted alkyl, optionally substituted aryl, or optionally substituted heteroaryl; and
   R¹¹ is optionally substituted alkyl, optionally substituted aryl, or optionally substituted heteroaryl; and
D is ═O, or
one or more of D and R¹ to R¹¹ is derivatized to facilitate incorporation into the medical device/material.

In some embodiments, the present invention pertains to a device/material employing a compound represented by Formula I, where:
R¹, R², R³ and R⁴ are chosen from hydrogen, halo (such as chloro and fluoro), hydroxy, lower alkyl (such as methyl), substituted lower alkyl, lower alkoxy (such as methoxy), and cyano;
R⁵ is optionally substituted aralkyl (such as benzyl or substituted benzyl);
R⁶ is hydrogen;
R⁷ is lower alkyl (such as ethyl, i-propyl, c-propyl or t-butyl);
R⁸ is substituted alkyl (such as a primary-, secondary- or tertiary-amino-substituted lower alkyl); and
R⁹ is —C(O)—R¹⁰ or —C(O)₂—R¹¹, in which:
   R¹⁰ is: optionally substituted alkyl (such as lower alkoxyalkyl), optionally substituted aryl (such as phenyl, such as lower alkyl-, hydroxy lower alkyl-, lower alkoxy-, and/or halo-substituted phenyl), optionally substituted aralkyl (such as optionally substituted benzyl and phenylvinyl), aryloxyalkyl (such as phenoxy lower alkyl), optionally substituted heteroaryl, optionally substituted heteroaralkyl, or optionally substituted heteroaryloxyalkyl; or
   R¹¹ is: optionally substituted aryl (such as phenyl, preferably lower alkyl-, lower alkoxy-, and/or halo-substituted phenyl) or optionally substituted heteroaryl.
      In some embodiments, the compounds of Formula 1 are chosen from:

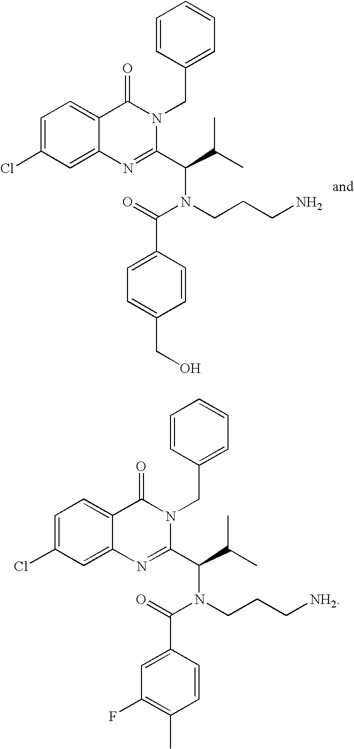

In some embodiments, the invention provides a drug delivery device incorporating an effective amount of rapamycin and at least one KSP inhibitor, such as at least one chemical entity chosen from compound represented by Formula I and pharmaceutically acceptable salts thereof.

Still another aspect of the invention entails a method of treating a mammal suffering from a cellular proliferative disease or a disorder that can be treated by modulating KSP activity, by administering a therapeutically effective amount of at least one KSP inhibitor, such as at least one chemical entity chosen from compounds represented by Formula I and pharmaceutically acceptable salts thereof via introduction of a medical device or material into or onto the body of such mammal.

Other aspects and embodiments will be apparent to those skilled in the art from the following detailed description.

As used in the present specification, the following words and phrases are generally intended to have the meanings as set forth below, except to the extent that the context in which they are used indicates otherwise.

“Incorporation” of a compound into a medical device, material or a coating thereon, means that the compound is associated with, bound, part of, entrapped or contained within the device, material or coating, whether physically or chemically, in such a manner as to facilitate controlled and/or sustained release of the compound in situ.

“Medical device” means an article of manufacture adapted for placement into the body of a mammal. The devices of the present invention, in addition to their customary function, also provide for the in situ delivery of a therapeutically effective amount of a compound or salt of Formula I. Such medical devices include, for example: subcutaneous implants, stents, angioplasty balloons, contact lenses, brachytherapy seeds, orthopedic and dental bone dowels, and prostheses such as breast implants, surgical pins, artificial joints, heart valves and vessels. The term “medical device” does not encompass syringes or unit dosage forms such as pills, capsules, suppositories or the like.

“Medical material” means an article of manufacture adapted for use in providing treatment to a mammal (such as in a surgical or dental procedure, or in the administration of first aid). The materials of the present invention, in addition to their customary function, also provide for the in situ delivery of a therapeutically effective amount of a compound or salt of Formula I. Such medical materials include, for example: surgical sponges, wound dressings, sheets, coatings, and solid- or semi-solid-forming fluids for introduction into body cavities. The term “medical material” does not encompass pharmaceutical formulations such as parenteral or intravenous liquid injectables, oral suspensions, perfusion fluids or the like.

“Medical device/material” means a medical device or a medical material.

“Polymer component” means a monomer, co-monomer, co-monomer mixture, polymer or co-polymer portion of a medical device/material.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl,” as defined below. It will be understood by those skilled in the art with respect to any group containing one or more substituents that such groups are not intended to introduce any substitution or substitution patterns that are sterically impractical, synthetically non-feasible and/or inherently unstable.

“Alkyl” is intended to include linear, branched, or cyclic hydrocarbon structures and combinations thereof. Lower alkyl refers to alkyl groups of from 1 to 5 carbon atoms. Examples of lower alkyl groups include methyl, ethyl, propyl, isopropyl, butyl, s- and t-butyl and the like. Preferred alkyl groups are those of C₂₀ or below. More preferred alkyl groups are those of C₁₃ or below. Still more preferred alkyl groups are those of C₆ and below. Cycloalkyl is a subset of alkyl and includes cyclic hydrocarbon groups of from 3 to 13 carbon atoms. Examples of cycloalkyl groups include c-propyl, c-butyl, c-pentyl, norbornyl, adamantyl and the like. In this application, alkyl refers to alkanyl, alkenyl and alkynyl residues; it is intended to include cyclohexylmethyl, vinyl, allyl, isoprenyl and the like. Alkylene is another subset of alkyl, referring to the same residues as alkyl, but having two points of attachment. Examples of alkylene include ethylene (—CH₂CH₂—), propylene (—CH₂CH₂CH₂—), dimethylpropylene (—CH₂C(CH₃)₂CH₂—) and cyclohexylpropylene (—CH₂CH₂CH(C₆H₁₃)—). When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl.

The term “alkoxy” or “alkoxyl” refers to the group —O-alkyl, preferably including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

The term “substituted alkoxy” refers to the group —O-(substituted alkyl). One preferred substituted alkoxy group is “polyalkoxy” or —O-(optionally substituted alkylene)-(optionally substituted alkoxy), and includes groups such as —OCH₂CH₂OCH₃, and glycol ethers such as polyethyleneglycol and —O(CH₂CH₂O)ₓCH₃, where x is an integer of about 2-20, preferably about 2-10, and more preferably about 2-5. Another preferred substituted alkoxy group is hydroxyalkoxy or —OCH₂(CH₂)_{y}OH, where y is an integer of about 1-10, preferably about 1-4.

“Acyl” refers to groups of from 1 to 10 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. “Lower-acyl” refers to groups containing 1 to 4 carbons and “acyloxy” refers to the group O-acyl.

The term “amino” refers to the group —NH₂. The term “substituted amino” refers to the group —NHR or —NRR where each R is independently selected from the group: optionally substituted alkyl, optionally substituted alkoxy, optionally substituted amino, optionally substituted aryl, optionally substituted heteroaryl, optionally substituted heterocyclyl, acyl, alkoxycarbonyl, sulfanyl, sulfinyl and sulfonyl, e.g., diethylamino, methylsulfonylamino, furanyl-oxy-sulfonamino.

“Aryl” and “heteroaryl” mean a 5-, 6- or 7-membered aromatic or heteroaromatic ring containing 0-4 heteroatoms selected from O, N or S; a bicyclic 9- or 10-membered aromatic or heteroaromatic ring system containing 0-4 (or more) heteroatoms selected from O, N or S; or a tricyclic 12- to 14-membered aromatic or heteroaromatic ring system containing 0-4 (or more) heteroatoms selected from O, N or S. The aromatic 6- to 14-membered aromatic carbocyclic rings include, e.g., phenyl, naphthalene, indane, tetralin, and fluorene and the 5- to 10-membered aromatic heterocyclic rings include, e.g., imidazole, oxazole, isoxazole, oxadiazole, pyridine, indole, thiophene, benzopyranone, thiazole, furan, benzimidazole, quinoline, isoquinoline, quinoxaline, pyrimidine, pyrazine, tetrazole and pyrazole.

“Aralkoxy” refers to the group —O-aralkyl. Similarly, “heteroaralkoxy” refers to the group —O-heteroaralkyl; “aryloxy” refers to —O-aryl; and “heteroaryloxy” refers to the group —O-heteroaryl.

“Aralkyl” refers to a residue in which an aryl moiety is attached to the parent structure via an alkyl residue. Examples include benzyl, phenethyl, phenylvinyl, phenylallyl and the like. “Heteroaralkyl” refers to a residue in which a heteroaryl moiety is attached to the parent structure via an alkyl residue. Examples include furanylmethyl, pyridinylmethyl, pyrimidinylethyl and the like.

“Halogen” or “halo” refers to fluorine, chlorine, bromine or iodine. Fluorine, chlorine and bromine are preferred. Dihaloaryl, dihaloalkyl, trihaloaryl etc. refer to aryl and alkyl substituted with a plurality of halogens, but not necessarily a plurality of the same halogen; thus 4-chloro-3-fluorophenyl is within the scope of dihaloaryl.

“Heterocycle” means a cycloalkyl or aryl residue in which one to four of the carbons is replaced by a heteroatom such as oxygen, nitrogen or sulfur. Examples of heterocycles that fall within the scope of the invention include imidazoline, pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, oxadiazole, dioxane, tetrahydrofuran and the like. “N-heterocyclyl” refers to a nitrogen-containing heterocycle as a substituent residue. The term heterocyclyl encompasses heteroaryl, which is a subset of heterocyclyl. Examples of N-heterocyclyl residues include 4-morpholinyl, 4-thiomorpholinyl, 1-piperidinyl, 1-pyrrolidinyl, 3-thiazolidinyl, piperazinyl and 4-(3,4-dihydrobenzoxazinyl). Examples of substituted heterocyclyl include 4-methyl-1-piperazinyl and 4-benzyl-1-piperidinyl.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(.±.)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)— or (S)—. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “pharmaceutically acceptable salt” refers to salts that retain the biological effectiveness and properties of the compounds of this invention and, which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

“Substituted-” alkyl, aryl, heteroaryl and heterocyclyl refer respectively to alkyl, aryl, heteroaryl and heterocyclyl wherein one or more (up to about 5, preferably up to about 3) hydrogen atoms are replaced by a substituent independently selected from the group: optionally substituted alkyl (e.g., fluoroalkyl), optionally substituted alkoxy, alkylenedioxy (e.g. methylenedioxy), optionally substituted amino (e.g., alkylamino and dialkylamino), optionally substituted amidino, optionally substituted aryl (e.g., phenyl), optionally substituted aralkyl (e.g., benzyl), optionally substituted aryloxy (e.g., phenoxy), optionally substituted aralkoxy (e.g., benzyloxy), carboxy (—COOH), carboalkoxy (i.e., acyloxy or —OOCR), carboxyalkyl (i.e., esters or —COOR), carboxamido, aminocarbonyl, benzyloxycarbonylamino (CBZ-amino), cyano, carbonyl, halogen, hydroxy, optionally substituted heteroaryl, optionally substituted heteroaralkyl, optionally substituted heteroaryloxy, optionally substituted heteroaralkoxy, nitro, sulfanyl, sulfinyl, sulfonyl, and thio.

The term “sulfanyl” refers to the groups: —S-(optionally substituted alkyl), —S-(optionally substituted aryl), —S-(optionally substituted heteroaryl), and —S-(optionally substituted heterocyclyl).

The term “sulfinyl” refers to the groups: —S(O)—H, —S(O)-(optionally substituted alkyl), —S(O)-(optionally substituted amino), —S(O)-(optionally substituted aryl), —S(O)-(optionally substituted heteroaryl), and —S(O)-(optionally substituted heterocyclyl).

The term “sulfonyl” refers to the groups: —S(O₂)—H, —S(O₂)-(optionally substituted alkyl), —S(O₂)-(optionally substituted amino), —S(O₂)-(optionally substituted aryl), —S(O₂)-(optionally substituted heteroaryl), —S(O₂)-(optionally substituted heterocyclyl), —S(O₂)-(optionally substituted alkoxy), —S(O₂)-optionally substituted aryloxy), —S(O₂)-(optionally substituted heteroaryloxy), and —S(O₂)-(optionally substituted heterocyclyloxy).

The term “therapeutically effective amount” or “effective amount” refers to that amount of a compound or salt of Formula I that is sufficient to effect treatment, as defined below, when administered to a mammal in need of such treatment. The therapeutically effective amount will vary depending upon the subject and disease condition being treated, the weight and age of the subject, the severity of the disease condition, the particular compound of Formula I chosen, the dosing regimen to be followed, timing of administration, the manner of administration and the like, all of which can readily be determined by one of ordinary skill in the art.

The term “treatment” or “treating” means any treatment of a disease in a mammal, including:
a) preventing the disease, that is, causing the clinical symptoms of the disease not to develop;
b) inhibiting the disease, that is, slowing or arresting the development of clinical symptoms; and/or
c) relieving the disease, that is, causing the regression of clinical symptoms.

In some embodiments, the present invention is directed to the medical devices/materials having support means (e.g., the structural framework of the device itself, a reservoir within such structural framework, a coating applied to the device, a polymer matrix or the like) adapted for introduction into or onto the body of a patient and incorporating a therapeutically effective amount of at least one KSP inhibitor, such as at least one chemical entity chosen from compounds represented by Formula I and pharmaceutically acceptable salts thereof. Such medical devices include, for example: subcutaneous implants, stents, angioplasty balloons, contact lenses, brachytherapy seeds, orthopedic and dental bone dowels, and prostheses such as breast implants, surgical pins, artificial joints, heart valves and vessels. Medical materials include, for example: surgical sponges, wound dressings, sheets and coatings (where the material's structural framework can be a drug-incorporating polymer matrix), and solid- or semi-solid-forming fluids for introduction into body cavities.

The KSP inhibitor can be incorporated directly within the body (or skeleton) of the device or material itself, for example in a reservoir, or in micropores or channels, or can be covalently bound (via solution chemistry techniques, such as the Carmeda process) or dry chemistry techniques (via vapor deposition methods such as rf-plasma polymerization) to the device or material itself. Alternatively, the KSP inhibitor can be incorporated into a coating that is later applied to the medical device or material, or deposited on a surface and then covered with a selectively permeable or biodegradable coating. The devices and materials are fabricated from biocompatible materials (e.g., non-reactive metals, polymers and the like), all or some of which can optionally, depending on intended use, be biodegradable.

In some embodiments, the present invention provides a vascular stent for use in PTCA, incorporating a KSP inhibitor in a polymer coating or in a reservoir provided with a coating or membrane for precisely delivering said KSP inhibitor at a predetermined rate.

In some embodiments, for example, a heart valve or a synthetic vessel, a KSP inhibitor can be incorporated directly into the polymeric matrix from which the device (or a portion thereof) is fabricated.

In some embodiments (for example, to be used in perivascular wrapping around grafted vessels or organs at the point of anastomosis) a KSP inhibitor is incorporated into a polymeric matrix that serves as the structural framework of the material to form a drug-impregnated sheet having a thickenss of about 10μ to 1000μ.

The medical devices and materials of the invention utilize at least one KSP inhibitor, such as at least one chemical entity chosen from compounds represented by Formula I: 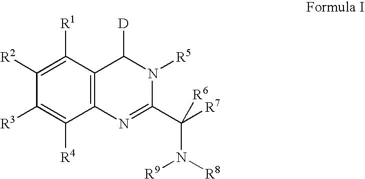
and pharmaceutically acceptable salts thereof, where:
R¹, R², R³ and R⁴ are independently hydrogen, hydroxy, halo, optionally substituted alkyl, optionally substituted alkoxy, optionally substituted amino, optionally substituted aryl, acyl, nitro, or cyano;
R⁵ is optionally substituted alkyl or optionally substituted aryl;
R⁶ and R⁷ are independently hydrogen, optionally substituted alkyl or optionally substituted aryl;
R⁸ is optionally substituted alkyl or optionally substituted aryl;
R⁹ is hydrogen, —C(O)—R¹⁰, —CH₂—R¹⁰, —C(O)—NH—R¹⁰, —S(O)₂—NH—R¹⁰, —C(O)₂—R¹¹, or —S(O)₂—R¹¹, in which:
   R¹⁰ is hydrogen, optionally substituted alkyl, optionally substituted aryl, or optionally substituted heteroaryl; and
   R¹¹ is optionally substituted alkyl, optionally substituted aryl, or optionally substituted heteroaryl; and
D is ═O, or
one or more of D and R¹ to R¹¹ is derivatized to facilitate incorporation into the medical device/material.

In the compounds represented by Formula I where one or more of D and R¹ to R¹¹ is derivatized to facilitate incorporation into a medical device/material, such derivitization is primarily focused on interaction of the compound with a portion (e.g., a polymeric coating) of the device/material, and is additionally selected to modulate the release kinetics for the active agent of Formula I. In some embodiments, the substituents R⁵ to R⁹, particularly R⁶ to R⁹, and most preferably R⁸ and/or R⁹ are derivatized. For example, incorporation with a biodegradable polymer matrix can be facilitated by a stable covalent bond where degradation of the polymer will affect release of the active agent from the medical device/material. A hydrolytically or enzymatically labile covalent bond would be more suitable to facilitate incorporation with a non-biodegradable polymer component where degradation of the bond will affect release of the active agent from the medical device/material, or even release from a biodegradable polymer component in the environment of a cell targeted for therapeutic modulation of KSP. At least one chemical entity described herein and further having a portion that has been rendered hydrophilic will incorporate with a hydrophilic polymer component; the converse is applicable to hydrophobic polymer component. The choice of pharmaceutically acceptable salt can likewise be tailored for retention and release kinetics with the medical device/material.

More particularly, incorporation of at least one chemical entity chosen from compounds represented by Formula I and pharmaceutically acceptable salts thereof into a medical device/material can be facilitated by such derivatizations as:
A hydrolysable bond to a polymer component of the medical device/material (particularly an acetal, amide, aminal, ester, imine, phosphate ester or Si moiety susceptible to cleavage under slightly acidic or enzymatic conditions), such as:
   R⁵ to R⁹, such as R⁶ to R⁹, for example, R⁸ and/or R⁹ being a hydroxyl substituent on a phenyl or aliphatic moiety.
   D being acryloylimino, 2-methyl-acryloylimino, trimethylsilanyloxy, or 3-(acrylamino)propyl-dimethylsilanyloxy.
   One of R¹ to R⁴ being hydroxy.
   R⁵ being p-acroyl-benzyl, p-methacroyl-benzyl or 2-hydroxypropionyl-benzyl.
   R⁸ being 3-(2-hydroxy-propionylamino)-propyl, e.g., as described in U.S. 2003/0008971 A1 where a combination of hydrophilic and hydrophobic co-macromers (i.e., co-monomers having a weight average molecular weight ranging from 500 to 80,000) are crosslinked to form a polymer network structure. In some embodiments, the hydrophilic macromers contain hydroxyl moieties (particularly polysaccharides, especially dextran). In some embodiments, the hydrophobic macromers contain unsaturated (e.g., vinyl) moieties [particularly poly(lactic acid) where a terminal carboxylic acid group has been converted to an aminoethanol group].
A positive or negative ionic charge complementary to a charged portion of the medical device/material.
Biotin with a matrix that allows protein adsorption.
An antibody/antigen interaction.
A pro-drug type coupling, e.g., as described in Ettmayer et al., J. Med. Chem., 2004, Vol. 47, No. 10, 2393-2404.

Other KSP inhibitors useful in the practice of the invention include those disclosed in U.S. Pat. Nos. 6,545,004, 6,562,831 and 6,630,479; in U.S. patent application Ser. No. 10/982,195, filed Nov. 5, 2004; and in PCT Applications WO01/30768, WO01/98278, WO02/56880, WO02/57244, WO03/39460, WO03/49527, WO03/49678, WO03/49679, WO03/50064, WO03/50122, WO03/79973, WO03/99211, WO03/103575, WO04/04652, WO04/06865, WO04/09036, WO04/18058, WO04/24086, WO04/32840, WO04/32879, WO04/34972, WO04/88903, WO04/94839, WO04/97053, PCT/US03/30788, each incorporated herein by reference, including derivitizations thereof (such as those described above) to facilitate the incorporation of these KSP inhibitors into a medical device/material.

The compounds of Formula I can be named and numbered (e.g., using AutoNom version 2.1) as described below. For example, the compound of Formula IA: 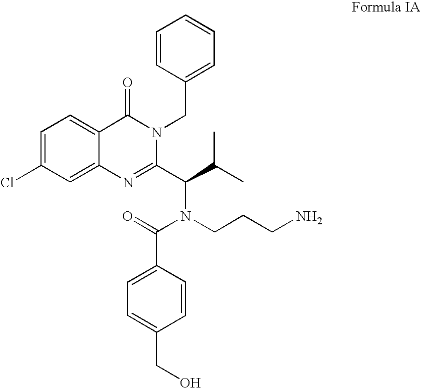
i.e., the compound according to Formula I where R¹, R², R⁴ and R⁶ are hydrogen, R³ is chloro, R⁵ is benzyl, R⁷ is (R)-iso-propyl, R⁸ is 3-aminopropyl, and R⁹ is —C(O)—R¹⁰ where R¹⁰ is 4-hydroxymethylphenyl can be named (R)-N-(3-amino-propyl)-N-[1-(3-benzyl-7-chloro-4-oxo-3,4-dihydro-quinazolin-2-yl)-2-methyl-propyl]-4-hydroxymethyl-benzamide.

Formulae IB throrugh IF illustrate compounds where one or more of D and R¹ to R¹¹ has been derivatized to facilitate incorporation into a medical device/material; all are shown illustrating the (R)— stereoisomer for substituent R⁷, but, stereochemical nomenclature is not recited in the following names for the sake of brevity. For example, Formula IB corresponds to Formula IA, in which R⁹ is —C(O)—R¹⁰ where R¹⁰ has been derivatized as a methacrylic acid. 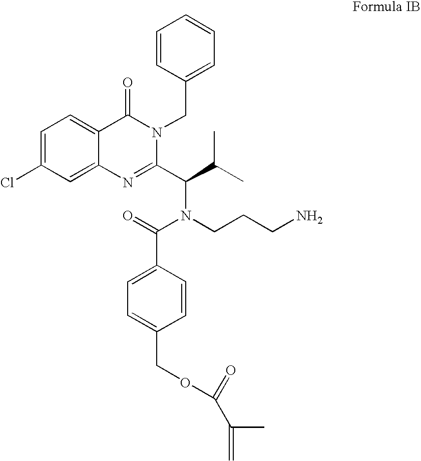
The compound of Formula IB can be named 2-methyl-acrylic acid 4-{(3-amino-propyl)-[1-(3-benzyl-7-chloro-4-oxo-3,4-dihydro-quinazolin-2-yl)-2-methyl-propyl]-carbamoyl}-benzyl ester.

In the compound of Formula IC, R⁹ is —C(O)—R¹⁰ where R¹⁰ has been derivatized as a phosphoric acid. 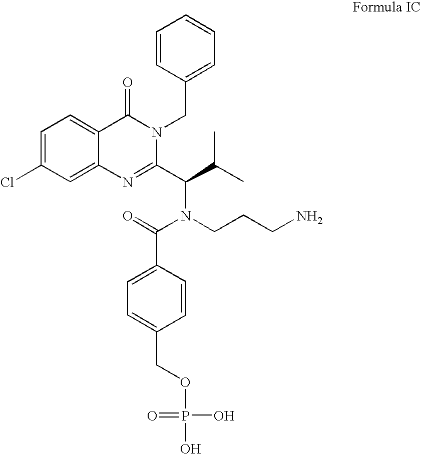
This compound can be named phosphoric acid mono-(4-{(3-amino-propyl)-[1-(3-benzyl-7-chloro-4-oxo-3,4-dihydro-quinazolin-2-yl)-2-methyl-propyl]-carbamoyl}-benzyll) ester.

In the compound of Formula ID, the substituent D has been derivatized as an acryloylimide. 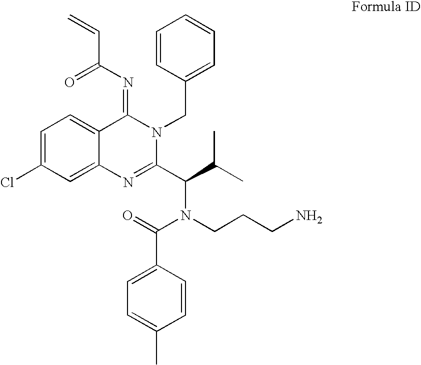
This compound can be named N-[1-(4-Acryloylimino-3-3-benzyl-7-chloro-3,4-dihydro-quinazolin-2-yl)-2-methyl-propyl]-N-(3-amino-propyl)-4-methyl-benzamide.

In the compound of Formula IE, the substituent D has been derivatized as a trimethylsilox alkyl acryloylimide. 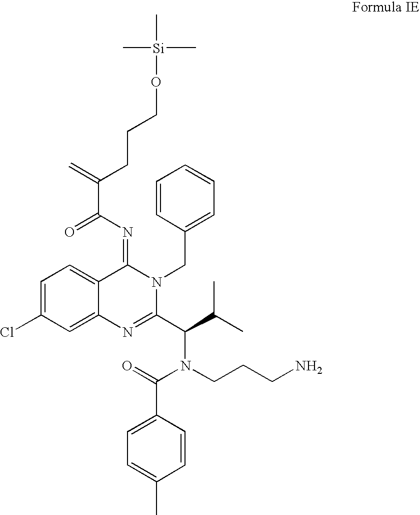
This compound can be named N-(3-Amino-propyl)-N-(1-{3-benzyl-7-chloro-4-[2-(3-trimethylsilanyloxy-propyl)-acryloylimino]-3,4-dihydro-quinazolin-2-yl}-2-methyl-propyl]-4-methyl-benzamide.

In the compound of Formula IF, the substituent R has been derivatized as an acryolamide. 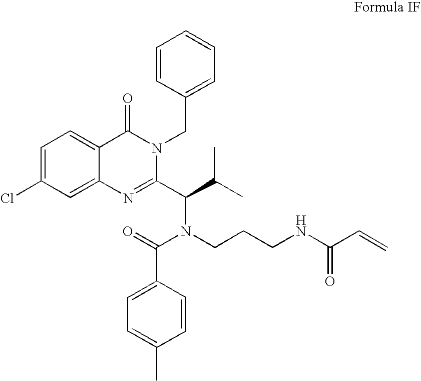
This compound can be named N-(3-acryloylamino-propyl)-N-[1-(3-benzyl-7-chloro-4-oxo-3,4-dihydro-quinazolin-2-yl)-2-methyl-propyl]-4-methyl-benzamide.

The compounds of the invention can be synthesized utilizing techniques well known in the art. See, for example, WO 01/30768 (incorporated herein by reference) where the methodology shown in Reaction Schemes A and B (below) is described. Stereospecific syntheses, e.g., employing D-valine as a starting material, are described in US-2004-0067969-A1 (also incorporated herein by reference) and illustrated in Reaction Scheme C. It will be appreciated by those skilled in the art that while Reaction Schemes A-C illustrate the synthesis of certain groups of compounds represented by Formula I (e.g., where R⁹ is —C(O)—R¹⁰) that the other compounds can be obtained by appropriate substitution of starting materials, reagents and/or reaction conditions.

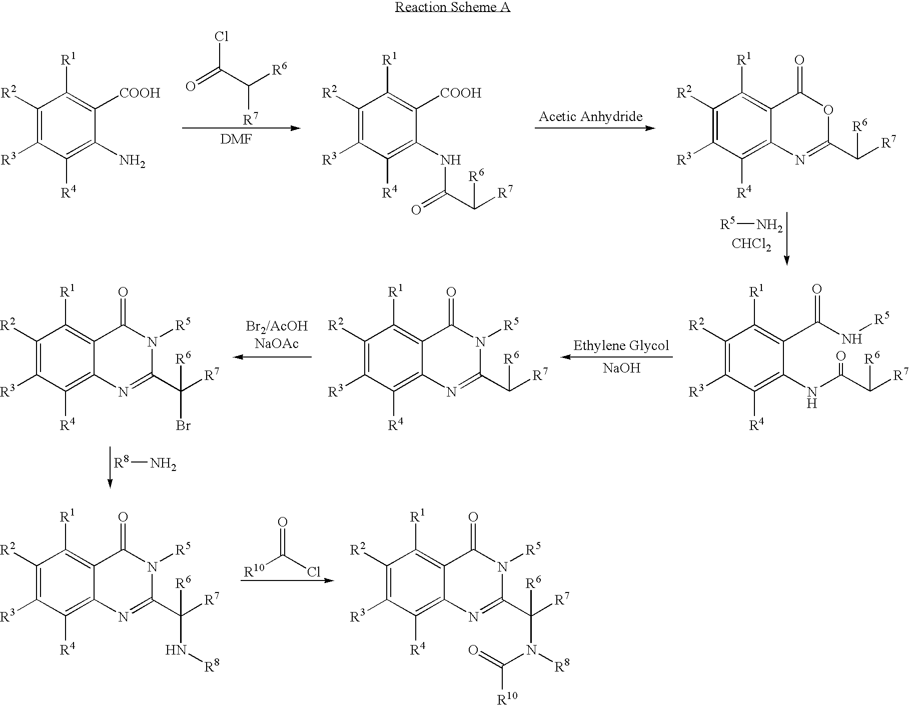

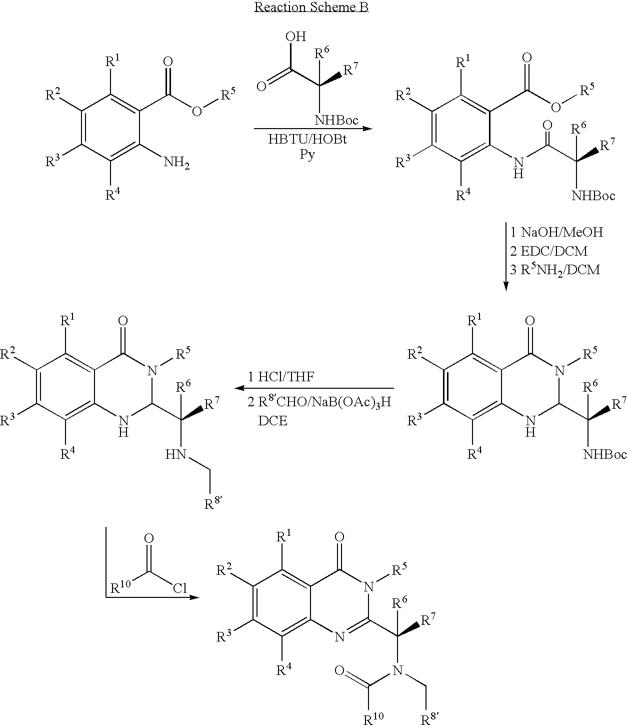

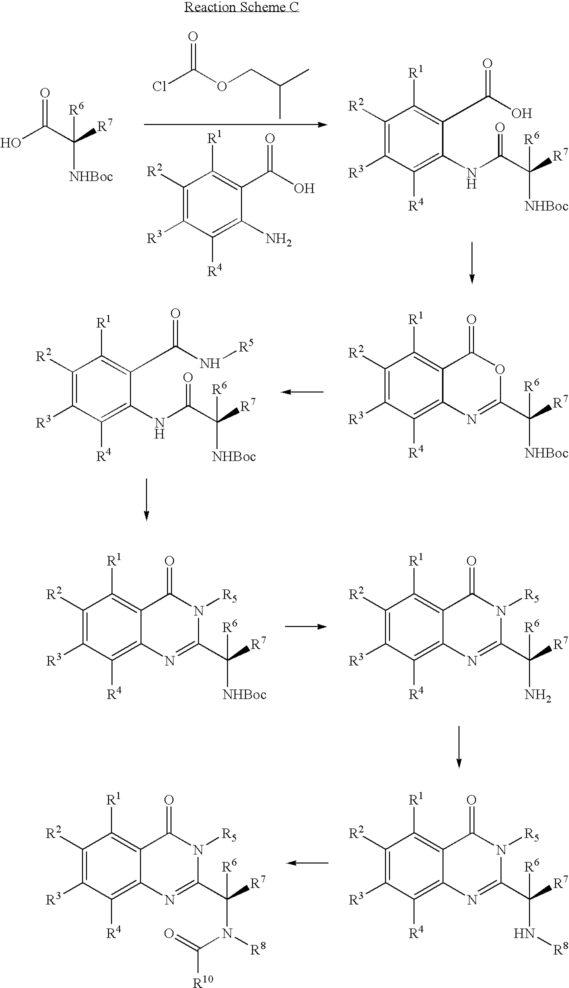

The compounds represented by Formula I where one or more of D and R¹ to R¹¹ is derivatized to facilitate incorporation into a medical device/material can be made by suitable substitution of the desired moieties in Reaction Schemes A, B and C. For example, by contacting an acryloyl halide with a compound of Formula I where R⁸ is aminoalkyl, or by protecting a para-hydroxyl or -phosphate of a tolyl halide and reacting it with the penultimate compound of Reaction Scheme B followed by deprotection to afford the hydroxy methyl benzamide or phosphoric acid of Formula I. A compound of Formula I having a free carboxylic acid can be conjugated to a biotyn-containing matrix by contact with DCC/HOBT and strepavidin in the presence of the matrix.

In some embodiments, the present invention pertains to a device/material employing a compound represented by Formula I where, for any of D and/or R¹ to R¹¹ that is not derivatized to facilitate incorporation into a medical device/material, the corresponding substituent is as follows.
D is ═O;
R¹, R², R³ and R⁴ are chosen from hydrogen, halo (such as chloro and fluoro), hydroxy, lower alkyl (such as methyl), substituted lower alkyl, lower alkoxy (such as methoxy), and cyano;
R⁵ is optionally substituted aralkyl (such as benzyl or substituted benzyl; for example, benzyl);
R⁶ is hydrogen;
R⁷ is lower alkyl (such as ethyl, i-propyl, c-propyl or t-butyl), particularly where R⁷ is the (R)-enantiomer;
R⁸ is substituted alkyl (such as a primary-, secondary- or tertiary-amino-substituted lower alkyl); and
R⁹ is —C(O)—R¹⁰ or —C(O)₂—R¹¹, in which:
   R¹⁰ is: optionally substituted alkyl (such as lower alkoxyalkyl), optionally substituted aryl (such as phenyl, lower alkyl-, hydroxy lower alkyl-, lower alkoxy-, and/or halo-substituted phenyl), optionally substituted aralkyl (such as optionally substituted benzyl and phenylvinyl), aryloxyalkyl (such as phenoxy lower alkyl), optionally substituted heteroaryl, optionally substituted heteroaralkyl, or optionally substituted heteroaryloxyalkyl; or
   R¹¹ is: optionally substituted aryl (such as phenyl, lower alkyl-, lower alkoxy-, and/or halo-substituted phenyl) or optionally substituted heteroaryl.
      The above-described groups and sub-groups are individually and collectively preferred, two or more being combined to describe further aspects of the invention.

Similarly, where one or more of D and/or R¹ to R¹¹ is derivatized to facilitate incorporation into a medical device/material, the preferred substituents for such derivitization are R⁵ to R⁹, particularly R⁶ to R⁹, and most preferably R⁸ and/or R⁹. Especially preferred derivitizations include hydrolytically or enzymatically labile covalent bonds (such as carboxylic and phosphoric acid esters), and acrylic cross-linking and/or co-polymerization.

In some embodiments, the compound of Formula 1 is chosen from
N-(3-amino-propyl)-N-[1-(3-benzyl-7-chloro-4-oxo-3,4-dihydro-quinazolin-2-yl)-2-methyl-propyl]-3-fluoro-4-methyl-benzamide;
N-(3-amino-propyl)-N-[1-(3-benzyl-7-chloro-4-oxo-3,4-dihydro-quinazolin-2-yl)-2-methyl-propyl]-4-hydroxymethyl-benzamide;
N-(3-amino-propyl)-N-[1-(3-benzyl-7-hydroxy-4-oxo-3,4-dihydro-quinazolin-2-yl)-2-methyl-propyl]-4-methyl-benzamide
N-(3-amino-propyl)-N-[1-(3-benzyl-7-chloro-4-oxo-3,4-dihydro-quinazolin-2-yl)-2-methyl-propyl]-4-methyl-benzamide phosphate ester;
2-methyl-acrylic acid 4-{(3-amino-propyl)-[1-(3-benzyl-7-chloro-4-oxo-3,4-dihydro-quinazolin-2-yl)-2-methyl-propyl]-carbamoyl}-benzyl ester;
phosphoric acid mono-(4-{(3-amino-propyl)-[1-(3-benzyl-7-chloro-4-oxo-3,4-dihydro-quinazolin-2-yl)-2-methyl-propyl]-carbamoyl}-benzyll) ester; and
N-(3-acryloylamino-propyl)-N-[1-(3-benzyl-7-chloro-4-oxo-3,4-dihydro-quinazolin-2-yl)-2-methyl-propyl]-4-methyl-benzamide.

In some embodiments, the compound of Formula 1 is chosen from
N-(3-amino-propyl)-N-[1-(3-benzyl-7-chloro-4-oxo-3,4-dihydro-quinazolin-2-yl)-2-methyl-propyl]-3-fluoro-4-methyl-benzamide; and
N-(3-amino-propyl)-N-[1-(3-benzyl-7-chloro-4-oxo-3,4-dihydro-quinazolin-2-yl)-2-methyl-propyl]-4-hydroxymethyl-benzamide.

Biodegradable (i.e., absorbable) polymer components suitable for use in the invention include:
lactone-based polyesters or copolyesters, e.g., poly(D,L-lactide), poly(D,L-lactide-co-glycolide), poly(carprolactone-glycolide);
poly(amino acids), poly(hydroxyvaleric acid), poly(malic acid), poly(tartronic acid)
polysaccharides, poly(co-glycolide), poly(glycolide), polyanhydrides, poly(alkylcarbonate, polydioxanone, polyphosphazenes;
polyesters, polypolyorthoesters, poly(ether-ester) copolymers, e.g., PEO-PLLA, poly(ethylene terephalate),
cellulose, such as methylcellulose, hydroxypropylcellulose, hydroxypropylmethylcellulose, carboxymethylcellulose, cellulose acetate phthalate, cellulose acetate succinate, and hydroxypropylmethylcellulose phthalate
albumin, collagen, gelatin, hyaluronic acid, starch, casein, dextrans, fibrinogen, and their copolymers.

Non-absorbable polymer components suitable for use in the invention include:
silicone rubber, polydimethylsiloxane;
polyethylene, polypropylene, poly(ethylene-vinylacetate) (“EVA”), polyethers such as poly(ethylene oxide), poly(propylene oxide), Pluronics and poly(tetramethylene glycol);
acrylic polymers, e.g., polyacrylic acid, polymethylacrylic acid, polymethyl methacrylate, poly(hydroxyethyl)methacrylate, polycyanoacrylate;
polyurethane, poly(ester urethanes), poly(ether urethanes), poly(ester urea)
vinyl polymers, e.g., polyvinylpyrrolidone (“PVP”), poly(vinyl alcohol), poly(vinyl acetate phthalate).
fluorinated polymers, e.g., polytetrafluoroethylene; and
cellulose esters, polyamides (nylon 6,6).

Ionic polymer components suitable for use in the invention include:
anionic polymers, e.g., alginate, carrageenan, carboxymethyl cellulose and poly(acrylic acid), and
cationic polymers, e.g., chitosan, poly-L-lysine, polyethyleneimine and poly(allyl amine).

Thermogelling polymer components (some listed with their gelling temperatures), suitable for use in the invention include: poly(N-methyl-N-n-propylacrylamide), 19.8° C.; poly(N-n-propylacrylamide), 21.5° C.; poly(N-methyl-N-isopropylacrylamide), 22.3° C.; poly(N, n-diethylacrylamide), 32.0° C.; poly(N-isopropylmethylacrylamide), 44.0° C.; poly(N-cyclopropylacrylamide), 45.5° C.; poly(N-ethylmethylacrylamide), 50.0° C.; poly(N-methyl-N-ethylacrylamide), 56.0; poly(N-cyclopropylmethacrylamide), 59.0° C.; and poly(N-ethylacrylamide), 72.0° C. Also included are co-polymers of the foregoing, as well as with other water-soluble polymers such as acrylmonomers (e.g., acrylic acid and derivatives thereof such as methacrylic acid, acrylate and derivatives thereof such as butyl methacrylate, acrylamide, and N-n-butyl acrylamide), cellulose ether derivatives (such as hydroxypropyl cellulose, 41° C.; methyl cellulose, 55° C.; hydroxypropylmethyl cellulose, 66° C.; and ethylhydroxyethylcellulose), Pluronics (such as F-127, 10-15° C.; L-122, 19° C.; L-92, 26° C.; L-81, 20° C.; and L-61, 24° C.) and polyoxyalkylene block copolymers.

Polymer components suitable for use with hydrophobic active agents of the invention include matrices of carbohydrates and polysaccharides such as starch, cellulose, dextran, methylcellulose, chitosan and hyaluronic acid, proteins or polypeptides such as albumin, collagen and gelatin.

As discussed in greater detail in U.S. Pat. No. 6,153,252, materials suitable for use as coatings to delay/sustain the release of active agents from the medical devices/materials of the invention are typically bioabsorbable or biostable film-forming polymers having melting temperatures above at least 40° C., preferably higher (e.g., above 55° C.). Bioabsorbable polymers include, for example: aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, biomolecules and blends thereof. Suitable film-forming biostable polymers with relatively low chronic tissue response include, for example: polyurethanes, silicones, poly(meth)acrylates, polyesters, polyalkyl oxides (polyethylene oxide), polyvinyl alcohols, polyethylene glycols and polyvinyl pyrrolidone, as well as, hydrogels such as those formed from crosslinked polyvinyl pyrrolidinone and polyesters. The bioabsorbable polymers are considered advantageous, for example, in that they present less risk of becoming dislodged over time.

Carrier matrices suitable for use, e.g., in dental and bone implants of the invention, are generally fibrous materials, such as textiles, filaments, cross-linked solid foams, gels and the like, providing microscopically dimensioned empty space allowing for hydration, efflux of drug and ingrowth of tissue, including: collagen, chemically cross-linked collagen or gelatin, cellulose, oxidized cellulose, cellulose acetate in fibrous form, ethyl cellulose, methyl cellulose, cellulose ethyl hydroxyethyl ether in fibrous form, poly-D,L-lactate, pyrolidone polymers in fibrous form, acrylic resins (e.g., polyacrylate, polymethacrylate, poly-hydroxybutyrate, poly-hydroxyvalerate, and their copolymers, in fibrous form), polyblycolic acid (Dexon), poly(D,L-lactic-co-glycolic acid), and polyglactin (Vicryl).

In some embodiments, polymer components suitable for use in the invention include poly(ethylene vinyl acetate), polyurethanes, poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(carpolactone) or poly(lactic acid) with polyethylene glycol (e.g., MePEG), polysaccharides such as hyaluronic acid, chitosan and funcans, and copolymers of polysaccharides with degradable polymers, and blends, admixtures, or copolymers of any of the above.

Biocompatible metals suitable for use in the invention include gold, silver, platinum, stainless steel, tantalum, and alloys typically used for such devices such as titanium alloys (including nitinol) and cobalt alloys (including cobalt-chromium-nickel alloys). Non-metallic biocompatible materials suitable for structural use include, for example: polyamides, polyolefins (e.g., polypropylene, polyethylene), non absorbable polyesters (e.g., polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone, etc. and blends thereof).

A polymer/drug coating can be applied to the surfaces of the device or material by dip-coating, spray coating, vapor deposition, brush coating, dip/spin coating and like techniques or combinations thereof. The drug/polymer ratio will be calculated based upon surface volume of the device/material, thickness and release characteristics of the coating, to achieve the desired loading. The solvents employed in the polymer-drug mixture are allowed to evaporate, leaving a film with the KSP inhibitor incorporated therein.

Drug devices and materials containing a KSP inhibitor within micropores, channels or one or more reservoirs are made by first forming the micropores/channels/reservoirs via the initial molding process or, e.g., by laser techniques. A solution is made of the KSP inhibitor in an organic solvent (e.g., acetone, methylene chloride), the concentration of which will be calculated in conjunction with micropore/channel/reservoir volume to achieve the desired loading. The device/material and the solution are contacted (optionally under compression) for a time sufficient to complete filling, and then removed. After evaporation of the solvent, the device/material is dipped briefly in fresh solvent to remove excess surface-bound drug. A solution of polymer coating material is applied by dip-coating, spray coating, vapor deposition, brush coating, dip/spin coating and like techniques or combinations thereof, to serve as release control means for delivering the KSP inhibitor.

For example, as discussed in U.S. Pat. No. 6,153,252 with regard to a coated stent, generally, the amount of polymer coating will vary with the polymer and the stent design and the desired effect of the coating, ranging from about 0.5% to about 20% (as a percent of the total weight of the stent after coating), preferably from about 1% to about 15%. The polymer coatings can be applied in one or more coating steps depending on the amount of polymer to be applied. A dilute first coating solution can advantageously be used as a primer to promote adhesion of a subsequent coating layers that may contain pharmaceutically active materials. A top coating can be applied to delay release of the pharmaceutical agent, or different coatings could be used as the matrix for the delivery of different pharmaceutically active materials. The amount of top coatings on a stent may vary, but will generally be less than about 2000 μg, preferably the amount of top coating will be in the range of about 10 μg to about 1700 μg and most preferably in the range of from about 300 μg to about 1600 μg. Different polymers can be used for different layers in the stent coating. Layering coatings of fast and slow hydrolyzing copolymers can be used to stage release of the drug or to control release of different agents placed in different layers. Polymer blends can also be used to control the release rate of different agents or to provide desirable balance of coating characterists (e.g., elasticity, toughness) and drug delivery characteristics (release profile). Polymers with different solubilities in various solvents can be employed to build up polymer layers to deliver different drugs or control the release profile of a drug. For example since ε-caprolactone-co-lactide elastomers are soluble in ethyl acetate and ε-caprolactone-co-glycolide elastomers are not soluble in ethyl acetate, a first layer of ε-caprolactone-co-glycolide elastomer containing a drug can be over coated with ε-caprolactone-co-lactide elastomer using a coating solution made with ethyl acetate as the solvent.

A KSP inhibitor can be incorporated directly into the polymeric material from which a medical device/material itself is fabricated by being mixed or solubilized with a skeleton polymer solution prior to fabrication, or dispersed into a skeleton polymer during fabrication, for example by extrusion, melt spinning, or molding.

Fabrication of a KSP inhibitor-bearing polymeric sheet can be accomplished by mixing or solubilizing a KSP inhibitor into a biodegradable and/or non-absorbable (co)polymer mixture followed by casting it as a thin sheet (e.g., 10μ to 1000μ thick).

Incorporation into the medical device/material can be accomplished, for example, as follows:
co-polymerizing of a Formula I/co-monomer with another co-monomer;
introducing a compound of Formula I into one or more reservoir(s) or micropores of the medical device/material. optionally followed by enveloping such coating with a selectively permeable membrane;
applying a coating of a compound of Formula I to the medical device/material, followed by enveloping such coating with a selectively permeable membrane;
introducing a compound of Formula I into a co-monomer mixture prior to polymerization, followed by application of the co-polymer/Formula I mixture to the medical device/material;
contacting a absorbable, polymer-coated medical device/material with a solution of a compound of Formula I, and optionally drying the medical device/material after absorption of a therapeutically effective amount of Formula I into the polymer coating.
applying a coating (such as Parylene C™) to the medical device/material followed by application (preferably by spraying) of a solution of co-monomers or co-polyers (such as PEVA and PBMA) and a compound of Formula I;

As will be appreciated by those in the art, mitosis may be altered in a variety of ways; that is, one can affect mitosis either by increasing or decreasing the activity of a component in the mitotic pathway. Stated differently, mitosis can be affected (e.g., disrupted) by disturbing equilibrium, either by inhibiting or activating certain components. Similar approaches may be used to alter meiosis.

The compounds and salts represented by Formula I can be used to modulate (i.e., increase or decrease) mitotic spindle formation, the organization of microtubules into bipolar structures. In this context, modulate means either increasing or decreasing spindle pole separation, causing malformation, i.e., splaying, of mitotic spindle poles, or otherwise causing morphological perturbation of the mitotic spindle. Mitotic spindle formation is mediated by mitotic kinesins. Compounds and salts of Formula I have been shown to bind to and/or modulate the activity of a mitotic kinesin, KSP (including variants and/or fragments of KSP) particularly human KSP, although modulation of other mitotic kinesins can be used in the present invention.

The medical devices and materials of the invention find use in a variety of applications including treatment of cellular proliferative diseases and disorders responsive to the modulation of KSP activity, including but not limited to, cancer, graft rejection, and proliferation induced after medical procedures, including, but not limited to, surgery, angioplasty, and the like. In some cases the targeted cells may not be in a hyper or hypo proliferation state (abnormal state) and still require treatment. For example, during wound healing, the cells may be proliferating “normally”, but proliferation enhancement may be desired.

The devices can be assessed in animal models relevant to the disease process one wishes to modify. For example, angioplasty and stent implantation in the blood vessels of pigs or rabbits can result in restenosis. Favorable modulation of this process by the implantation of a drug coated or eluting stent could be indicative of potential success in treating the disease process in humans. Ultimately, activity for treating heart disease is demonstrated in human clinical trials.

The medical devices and materials of the invention are typically placed/applied/used very much in the same manner as such devices and materials incorporating no active agent. They incorporate a therapeutically effective dosage of a compound or salt represented by Formula I, which will be dependent on the subject and disease state being treated, the severity of the affliction, the nature of the device/material, the rate and the duration of administration. For example, a daily dose for local delivery to prevent restenosis can be expected to be significantly lower and less dependent on body weight than a daily dose for the systemic treatment to prevent the recurrence of cancer. While human dosage levels have yet to be optimized, generally, a daily dose for local delivery to prevent restenosis can be estimated to be on the order of about 0.05 μg to 10 mg/day with a 30-day duration of treatment, resulting in a device loading on the order of 1.5 μg to 300 mg, again depending upon the active agent, device, subject, release kinetics and the like. Device loading for a dental bone dowel would need to provide an effective amount to encourage bone growth over a period of 6 to 8 months, again at a relatively small daily dosage.

### EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### Example 1

### Base-Coated Stent

A Paralene C™/active agent solution is made by dissolving 1.75 mg/ml poly(ethylene-covinyl acetate), 1.75 mg/ml polybutyl methacrylate, and 1.5 mg/ml N-(3-amino-propyl)-N-[1-(3-benzyl-7-chloro-4-oxo-3,4-dihydro-quinazolin-2-yl)-2-methyl-propyl]-3-fluoro-4-methyl-benzamide in 50 mL MTBE, with stirring at room temperature.

A stent is weighed and then mounted on a paralene-coating instrument (SCS—Madison, Wis.). The stent is coated with with the Paralene C™/active agent solution using a vapor deposition method provided by the manufacturer of the coating instrument. The coated stent is removed from the vapor spray and allowed to air-dry to afford a coated stent of the invention. The dried stent is re-weighed, the amount of Paralene C™/active agent coating is determined as the difference between pre- and post-coating weights, and the dosage of active agent is calculated.

### Example 2

### Dip-Coated Stent

An absorbable elastomer based on 45:55 mole percent copolymer of ε-caprolactone and blycolide, with an IV of 1.58 (0.1 g/dl in hexafluoroisopropanol at 25° C.) is dissolved 5% by weight in acetone to afford a low concentration coating material. The synthesis of the elastomer is described in U.S. Pat. No. 5,468,253, incorporated herein by reference.

Separately, a high concentration coating/active agent material is made as described above, dissolving 15% by weight of the 45:55 mole percent copolymer and 6% by weight of the active agent N-(3-amino-propyl)-N-[1-(3-benzyl-7-chloro-4-oxo-3,4-dihydro-quinazolin-2-yl)-2-methyl-propyl]-3-fluoro-4-methyl-benzamide in 1,1,2-trichloroethane.

A Cordis P-S 153 stent (commercially available from Cordis, a Johnson & Johnson company) is placed on a 0.032 in. (0.81 mm) diameter mandrel and immersed in a dip bath containing the low concentration coating material, to deposit an initial primer coat on the stent. The mandrel, with the stent on it, is removed from the dip bath and before the coating has a chance to dry the stent is moved along the length of the mandrel in one direction. This wiping motion applies high shear to the coating trapped between the stent and the mandrel. The high shear rate forces the coating out through slots cut into the tube from which the stent is formed. This wiping action serves to force the coating out of the slots and keep them clear. The “primed stent” is allowed to air dry at room temperature, and incorporates about 100 micrograms of coating.

After 2 hours of drying, the stent is re-mounted on a 0.0355 in. (0.9 mm) diameter clean mandrel and immersed in a dip bath containing the high concentration coating/active agent. The dip and wipe process is repeated. The “final coated stent” is air dried for 12 hours and then put in a 60° C. vacuum oven (at 30 in. Hg vacuum) for 24 hours to dry, affording a coated stent of the invention having about 270 micrograms of polymer and about 180 micrograms of N-(3-amino-propyl)-N-[1-(3-benzyl-7-chloro-4-oxo-3,4-dihydro-quinazolin-2-yl)-2-methyl-propyl]-3-fluoro-4-methyl-benzamide.

### Example 3

### In Vitro Drug Release Assay

Coated stents with known concentrations of active agent are prepared as described in Examples 1 and 2. Each stent is placed in 2.5 mL of release medium (aqueous ethanol; 15% by volume at room temperature) contained in a 13×100 mm culture tube. The tube is shaken in a water bath (INNOVA™ 3100; New Brunswick Scientific) at 200 rpm while maintaining ambient conditions. After a 1 hour, the tubes are removed from the shaker and the stents are carefully transferred to fresh 2.5 mL aliquots of release medium in clean tubes, respectively, which are placed back in the water bath on the shaker. The release media are reserved for subsequent analysis. Shaking is resumed for an additional hour, followed by stent removal and transfer to fresh release medium, as described above. After five removal and transfer steps, the stents are placed in a sixth aliquot of release medium, placed back in the water bath, and shaking is resumed until 24 hours following initial immersion. The stents are removed and reserved for physical inspection.

From the reserved aliquots of release medium, 20 μL samples are withdrawn and analyzed by HPLC on a C₁₈-reverse phase column (Waters Summetry™ Column: 4.6 mm×100 mm RP₁₈ 3.5 μm with a matching guard column) using a mobile phaswe consisting of acetonitrile/methanol/water (38:34:28 v/v) delivered at a flow rate of 1.2 mL/min in a Waters Alliance with a PDA 996, equipped with a photodiode array detector. The column is maintained at 60° C. through the analysis. The concentration of N-(3-amino-propyl)-N-[1-(3-benzyl-7-chloro-4-oxo-3,4-dihydro-quinazolin-2-yl)-2-methyl-propyl]-3-fluoro-4-methyl-benzamide in each aliquot is determined from a standard curve of concentration versus response (area under the curve) generated from standards in the range of 50 ng/mL to 50 μg/mL.

The active agent-coated stents of the invention demonstrate continuous delivery of active agent into the release medium over the test period.

### Example 4

### In Vivo Drug Release Assay

Coated stents with known concentrations of active agent are prepared as described in Examples 1 and 2. Male Yorkshire pigs are started on oral aspirin (325 mg/day) 3 days prior to study initiation. Experimental groups of study animals are treated as follows.

The pig is anesthetized with xylazine (2 mg/kg, IM) ketamine (17 mg/kg, IM) and atropine (0.02 mg/kg, IM) and then intubated using standard procedure, and placed on flow-by oxygen with 1-2.5% volatile isoflurane for maintenance anesthesia via the endotrachial tube. Peripheral intravenous access is achieved by insertion of a 20 gauge Angiocath into the marginal ear vein; a 20 gauge arterial catheter is also placed in the ear for continuous blood pressure and heart rate monitoring. Upon confirmation of adequate depth of anesthesia, the right inguinal region is shaved, sterilized, and draped. Using aseptic technique through the rest of the procedure, a linear incision parallel to the femoral vessel is made and the subcutaneous tissues dissected to the level of the artery. After adequate exposure, the femoral artery is isolated proximally with umbilical tape and distally with a 3.0 silk tie for hemostasis. Using surgical scissors, an arteriotomy is made and an 8 Fr sheath inserted in the artery. Heparin (4,000 units) and bretylium (75 mg) are then administered intravenously after sheath insertion. Electrocardiogram, respiratory pattern and hemodynamics are continuously monitored.

A hockey stick guiding catheter is inserted via the femoral sheath and advanced to the left coronary ostium, whereupon left coronary cineangiography is performed. A single frame anteroposterior radiogram is developed and the luminal diameters of the left descending and circumflex arteries measured, in order to size the balloon-stent assembly for a prespecified balloon-to-artery ratio of approximately 1.1-1.2:1. Using guide catheter support and fluoroscopic guidance, a 0.014 in. guidewire is advanced into the lumen of the left anterior descending artery. Intracoronary stenting is performed by advancing a stent in mounted on a conventional angioplasty balloon into position in the mid-portion of the left anterior descending artery. The stent is deployed by inflating the mounting balloon to 8 atmospheres for 30 seconds. Upon confirmation of vessel patency, the balloon and guidewire are removed from the left anterior descending artery, and the identical procedure is performed in the left circumflex artery. Upon completion of stent delivery in the left circumflex artery, the balloon and guidewire are withdrawn. The guiding catheter and femoral arterial sheath are then removed, the femoral artery tied proximally with 3-0 silk suture for homeostasis and the inguinal incision is closed. After discontinuation of anesthesia, the pig is returned to colony housing, where daily aspirin (325 mg) is continued until euthanasia.

At a selected time after stent implantation, euthanasia is performed by overdose of pentobarbital administered IV. The chest is opened via a mid-sternal incision and the heart removed. Both the LAD and LCX are carefully dissected free of surrounding tissue. The stent is then dissected free of the arterial tissue and placed in a vial; the amount of active agent remaining in the stent is determined by a variation of the procedure described in Example 2.

When tested as described above, the active agent-coated stents of the invention demonstrate delivery of active agent in vivo.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

1. A medical device or material having an effective amount of at least one KSP inhibitor.

2. A medical device or material of claim 1 wherein the at least one KSP inhibitor is at least one chemical entity chosen from compounds represented by Formula I: 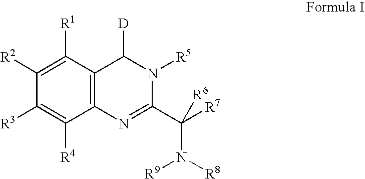
and pharmaceutically acceptable salts thereof, where:
R¹, R², R³ and R⁴ are independently hydrogen, hydroxy, halo, optionally substituted alkyl, optionally substituted alkoxy, optionally substituted amino, optionally substituted aryl, acyl, nitro, or cyano;
R⁵ is optionally substituted alkyl or optionally substituted aryl;
R⁶ and R⁷ are independently hydrogen, optionally substituted alkyl or optionally substituted aryl;
R⁸ is optionally substituted alkyl or optionally substituted aryl;
R⁹ is hydrogen, —C(O)—R¹⁰, —CH₂—R¹⁰, —C(O)—NH—R¹⁰, —S(O)₂—NH—R¹⁰, —C(O)₂—R¹¹, or —S(O)₂—R¹¹, in which:
R¹⁰ is hydrogen, optionally substituted alkyl, optionally substituted aryl, or optionally substituted heteroaryl; and
R¹¹ is optionally substituted alkyl, optionally substituted aryl, or optionally substituted heteroaryl; and
D is ═O, or
one or more of D and R¹ to R¹¹ is derivatized to facilitate incorporation into the medical device/material.

3. A medical device or material of claim 2 wherein R¹, R², R³ and R⁴ are chosen from hydrogen, chloro, fluoro), hydroxy, methyl, substituted lower alkyl, methoxy, and cyano.

4. A medical device or material of claim 2 wherein R⁵ is optionally substituted aralkyl.

5. A medical device or material of claim 4 wherein R⁵ is benzyl or substituted benzyl.

6. A medical device or material of claim 5 wherein R⁵ is benzyl.

7. A medical device or material of claim 2 wherein R⁶ is hydrogen.

8. A medical device or material of claim 2 wherein R⁷ is lower alkyl.

9. A medical device or material of claim 8 wherein R⁷ is ethyl, i-propyl, c-propyl or t-butyl.

10. A medical device or material of claim 2 wherein R⁸ is substituted alkyl.

11. A medical device or material of claim 10 wherein R⁸ is primary-, secondary- or tertiary-amino-substituted lower alkyl.

12. A medical device or material of claim 2 wherein R⁹ is —C(O)—R¹⁰ or —C(O)₂—R¹¹, in which:
R¹⁰ is: optionally substituted alkyl, optionally substituted aryl, optionally substituted aralkyl, aryloxyalkyl, optionally substituted heteroaryl, optionally substituted heteroaralkyl, or optionally substituted heteroaryloxyalkyl; and
R¹¹ is: optionally substituted aryl or optionally substituted heteroaryl.

13. A medical device or material of claim 12 wherein R¹⁰ is: lower alkoxyalkyl, phenyl, preferably lower alkyl-, hydroxy lower alkyl-, lower alkoxy-, and/or halo-substituted phenyl, optionally substituted benzyl, phenylvinyl, phenoxy lower alkyl, optionally substituted heteroaryl, optionally substituted heteroaralkyl, or optionally substituted heteroaryloxyalkyl.

14. A medical device or material of claim 12 wherein R¹¹ is: phenyl, preferably lower alkyl-, lower alkoxy-, and/or halo-substituted phenyl or optionally substituted heteroaryl.

15. A medical device or material of claim 2 wherein one or more of D and/or R¹ to R¹¹ is derivatized to facilitate incorporation into the medical device/material.

16. A medical device or material of claim 15 wherein R⁵ to R⁹ is derivatized to facilitate incorporation into the medical device/material.

17. A medical device or material of claim 16 wherein R⁶ to R⁹ is derivatized to facilitate incorporation into the medical device/material.

18. A medical device or material of claim 17 wherein R⁸ and/or R⁹ is derivatized to facilitate incorporation into the medical device/material.

19. A medical device or material of claim 1 wherein the at least one KSP inhibitor is chosen from
N-(3-amino-propyl)-N-[1-(3-benzyl-7-chloro-4-oxo-3,4-dihydro-quinazolin-2-yl)-2-methyl-propyl]-3-fluoro-4-methyl-benzamide;
N-(3-amino-propyl)-N-[1-(3-benzyl-7-chloro-4-oxo-3,4-dihydro-quinazolin-2-yl)-2-methyl-propyl]-4-hydroxymethyl-benzamide;
N-(3-amino-propyl)-N-[1-(3-benzyl-7-hydroxy-4-oxo-3,4-dihydro-quinazolin-2-yl)-2-methyl-propyl]-4-methyl-benzamide
N-(3-amino-propyl)-N-[1-(3-benzyl-7-chloro-4-oxo-3,4-dihydro-quinazolin-2-yl)-2-methyl-propyl]-4-methyl-benzamide phosphate ester;
2-methyl-acrylic acid 4-{(3-amino-propyl)-[1-(3-benzyl-7-chloro-4-oxo-3,4-dihydro-quinazolin-2-yl)-2-methyl-propyl]-carbamoyl}-benzyl ester;
phosphoric acid mono-(4-{(3-amino-propyl)-[1-(3-benzyl-7-chloro-4-oxo-3,4-dihydro-quinazolin-2-yl)-2-methyl-propyl]-carbamoyl}-benzyl 1) ester; and
N-(3-acryloylamino-propyl)-N-[1-(3-benzyl-7-chloro-4-oxo-3,4-dihydro-quinazolin-2-yl)-2-methyl-propyl]-4-methyl-benzamide.

20. A medical device or material of claim 19 wherein the at least one KSP inhibitor is chosen from
N-(3-amino-propyl)-N-[1-(3-benzyl-7-chloro-4-oxo-3,4-dihydro-quinazolin-2-yl)-2-methyl-propyl]-3-fluoro-4-methyl-benzamide; and
N-(3-amino-propyl)-N-[1-(3-benzyl-7-chloro-4-oxo-3,4-dihydro-quinazolin-2-yl)-2-methyl-propyl]-4-hydroxymethyl-benzamide.

21. A medical device or material of claim 1 wherein the medical device or material further comprises an effective amount of rapamycin.

22. A medical device or material of claim 1 wherein the medical device is chosen from: subcutaneous implants, stents, angioplasty balloons, contact lenses, brachytherapy seeds, orthopedic and dental bone dowels, and prostheses.

23. A medical device or material of claim 22 wherein the medical device or material is chosen from breast implants, surgical pins, artificial joints, heart valves and vessels.

24. A medical device or material of claim 1 wherein the medical materials is chosen from surgical sponges, wound dressings, sheets and coatings, and solid- or semi-solid-forming fluids for introduction into body cavities.

25. A medical device or material of claim 1 wherein the medical device or material is a vascular stent for use in PTCA, incorporating the at least one KSP inhibitor in a polymer coating or in a reservoir provided with a coating or membrane for precisely delivering the at least one KSP inhibitor at a predetermined rate.

26. A medical device or material of claim 1 wherein the medical device or material is a heart valve or a synthetic vessel wherein the at least one KSP inhibitor is incorporated directly into the polymeric matrix from which the device or a portion thereof is fabricated.

27. A medical device or material of claim 1 wherein the at least one KSP inhibitor is incorporated into a polymeric matrix that serves as the structural framework of the medical material to form a drug-impregnated sheet having a thickness of about 10μ to 1000μ.

28. A method of treating a mammal suffering from a cellular proliferative disease or a disorder that can be treated by modulating KSP activity, by administering a therapeutically effective amount of at least one KSP inhibitor via introduction of a medical device or material into or onto the body of such mammal.

29. A method of claim 28 wherein the medical device or material is a medical device or material of claim 2.

30. A method of claim 28 wherein the cellular proliferative disease or disorder that can be treated by modulating KSP activity is chosen from atherosclerosis and restenosis.
